(19) United States
(12) Patent Application Publication
(10) Pub. No.: US 2018/0272395 A1
(43) Pub. Date: Sep. 27, 2018

(54) A Method of Processing Waste Material Including A Super Absorbent Polymer

(71) Applicant: PERSONNEL HYGIENE SERVICES LIMITED, Caerphilly Mid Glamorgan (GB)

(72) Inventor: Lee Herriott, Caerphilly Mid Glamorgan (GB)

(21) Appl. No.: 15/763,737

(22) PCT Filed: Oct. 3, 2016
(86) PCT No.: PCT/GB2016/053061

§ 371 Date: Mar. 27, 2018

(30) Foreign Application Priority Data
Oct. 1, 2015 (GB) 1517370.1

(51) Int. Cl. B09B 3/00; A61L 11/00

(52) U.S. Cl. CPC B09B 3/0008; B09B 3/0016; B09B 3/0075; B09B 2220/00; A61L 11/00

(57) ABSTRACT

A method of processing waste material including a super absorbent polymer includes: shredding the waste material; applying a salt to the shredded waste material to deactivate the super absorbent polymer; dewatering the waste material following deactivation of the super absorbent polymer; applying a liquid biocide to the shredded waste material. The preferred salt is aluminium sulphate. The step of applying a liquid biocide to the shredded waste is provided after the step of dewatering the waste material in the preferred form.

### BACKGROUND OF THE INVENTION

### 1. The Field of the Invention

The present invention relates to a method of processing waste material including a super absorbent polymer, and in particular a method of forming a solid fuel from such material.

### 2. The Relevant Technology

Disposal of absorbent sanitary products such as disposable nappies or feminine hygiene pads is an ever increasing product for waste management services. The majority of waste absorbent sanitary products, when segregated relative to other waste, come from care facilities such as nursing homes, residential homes and children's nurseries, together with washroom facilities and hospital waste. In the UK alone around one million tonnes of waste of this type is estimated to be generated each year. The cost of handling and disposing of this waste is significant, with some estimates placing the cost to the UK of disposing of waste nappies at over £100 million per year. In addition to the disposal costs, the environmental impact of waste nappies is also significant.

Nappies and other absorbent sanitary products are classed as ‘offensive waste’, meaning non-clinical waste that is non-infectious and doesn't contain pharmaceutical or chemical substances, but may be unpleasant to anyone who comes into contact with it. The majority of absorbent sanitary waste such as nappies is currently disposed of by landfill or incineration.

Disposable nappies typically consist of an absorbent, anatomically shaped pad having a soft liner for comfort and enclosed within a waterproof cover. The liner that covers the absorbent pad is made of a plastic polymer such as polypropylene and provides a protective layer next to the wearer's skin. The protective liner is porous to allow liquid to flow to the absorbent core. The absorbent core is surrounded by a pulp fibre layer with the core itself comprising a super absorbent polymer (SAP) material that swells on contact with liquid as the liquid is absorbed within the SAP. The SAP is typically synthetic and may be a polyacrylate, polyacrylamide, or other hydrophilic component. The SAP is generally provided in a granular form. The SAP absorbs liquid including water, urine or other body fluids and forms a bond with the liquid, thereby retaining the liquid and preventing re-release or discharge from the core.

Currently residential waste disposal policy, in the UK at least, requires nappies to be disposed of through general ‘black bag’ waste. Hospitals either send absorbent sanitary waste for landfill or incinerate the waste on site. Currently councils do not have the facility to recycle nappy waste, and there are health implications to mixing nappies with other recycling waste. Nonetheless processes are currently being developed to recycle some of the material from waste nappies. Such processes typically involve shredding the nappies and saturating the SAP material with water before pressing the shredded material to separate the SAP and fibrous material from the plastic material. Following the pressing process a salt such as aluminium sulphate may be added to the material to deactivate the SAP. This prevents any SAP that remains with the plastic from swelling when the plastic has been recycled into a moulded product. In other processes an autoclave may be used to separate and clean the material through a process of heating, pressurisation, agitation and shredding.

Such processes are relatively energy intensive and only recover a limited amount of recyclable material from the waste.

It is therefore desirable to provide an improved means of processing waste products containing super absorbent polymers which addresses the above described problems and/or which offers improvements generally.

According to a first aspect of the present invention there is provided a method of processing waste products containing super absorbent polymers as described in the accompanying claims.

According to a second aspect of the invention there is provided a method of processing waste material including a super absorbent polymer, the method comprising:
a) shredding the waste material;
b) applying a liquid biocide to the shredded waste material;
c) applying a salt to the shredded waste material to deactivate the super absorbent polymer; and
d) dewatering the waste material following deactivation of the super absorbent polymer.

According to a third aspect of the invention there is provided a method of processing waste material including a super absorbent polymer, the method comprising:
a) shredding the waste material;
b) applying a salt to the shredded waste material;
c) dewatering the waste material following application of the salt;
d) applying a biocide to the shredded waste material.

According to a fourth aspect of the invention there is provided a method of processing waste material including a super absorbent polymer, the method comprising:
a) shredding the waste material;
b) applying a salt to the shredded waste material;
c) dewatering the waste material following application of the salt.

The first and/or second and/or third and/or fourth aspects of the invention may include any of the features, options or possibilities set out elsewhere in this application, including the following.

The waste material may include one or more of: incontinence products, nappies, diapers, sanitary towels, absorbent sanitary products pulp fibre bedpans, slipper pans and urinals. The waste material may be a mixture of two or more of: cellulose, cotton, plastics, super absorbent polymers, liquid, water, urine, menstrual discharge, faeces.

Preferably no separation of the plastics content of the waste material from the super absorbent polymer content of the waste material is provided or sought in the process. Preferably at least 80% of the plastics content of the waste material and at least 80% of the super absorbent polymer content of the waste material are present in the waste material entering the material collection stage and/or are present in the solid waste product stream from the process.

Preferably no separation of the plastics content of the waste material, the super absorbent polymer content of the waste material and the cotton and/or cellulose content of the waste material is provided or sought in the process. Preferably at least 80% of the plastics content of the waste material, at least 80% of the super absorbent polymer content of the waste material and at least 80% of the cellulose and/or content of the waste material are present in the waste material entering the material collection stage and/or are present in the solid waste product stream from the process.

The super absorbent polymer may be a polyacrylate, polyacrylamide, or other hydrophilic component. The super absorbent polymer may be provided in a granular or powder form

The process may include a waste material reception stage. The waste material reception stage may provide for one or more of: storing of waste material, opening containers for waste material, opening packaging for waste material, removing waste material from containers or packaging, providing waste material to a hopper or conveyor.

The method may include a metal screening stage. The waste material may be moved through the metal screening stage, for instance using a conveyor. The metal screening stage may include passing the waste material through a magnetic field, for instance provided by one or more magnets. The metal screening stage may remove magnetic materials from the waste material. The metal screening stage may be provided after a waste material reception stage. Preferably the metal screening stage is provided before the shredding stage.

Preferably no water or other liquid is added to the waste material between the start of the processing and the waste material entering the shredding stage. Preferably no water or other liquid is added to the waste material between the start of the processing and the waste material exiting the shredding stage. Preferably any water or other liquid added to the waste material between the start of the processing and the waste material entering the shredding stage is less than 10% by weight of the waste material, more preferably less than 5% by weight. Preferably no water or other liquid is added to the waste material between the start of the processing and the waste material exiting the shredding stage. Preferably any water or other liquid added to the waste material between the start of the processing and the waste material exiting the shredding stage is less than 10% by weight of the waste material, more preferably less than 5% by weight.

Preferably the moisture content of the waste material, excluding the moisture absorb by the super absorbent polymer in the waste material, is less than 25% by weight on entering the shredding stage. More preferably it is less than 20% and still more preferably less than 10%. Preferably the moisture content of the waste material, excluding the moisture absorb by the super absorbent polymer in the waste material, is less than 25% by weight on exiting the shredding stage. More preferably it is less than 20% and still more preferably less than 10%.

The shredding may be provided by a shredding stage. The shredding stage may include a rotating shredder, for instance a dual shaft shredder. The dual shaft shredder may provide two counter rotating shafts, with each shaft provided with a series of cutting blades. The shredding stage, particularly the rotating shredder, may control the maximum size of the shredded waste material, for instance through the spacing and size of the shafts and/or cutting blades. The maximum size of the shredded waste may be no greater than 150 mm, particularly no greater than 120 mm.

Preferably the shredding stage is conducted at the ambient temperature of the process environment +/−10° C., more preferably +/−5° C. Preferably the process and its stages are all conducted at the ambient temperature of the process environment +/−10° C., more preferably +/−5° C. Preferably no heating is inputted to the shredding stage. Preferably no heating is inputted to the waste material between entry to the process and exiting the process, for instance at the waste material collection and/or waste material wrapping stage.

The method may include a second metal screening stage. The waste material may be moved through the second metal screening stage, for instance using a conveyor. The second metal screening stage may include passing the waste material through a magnetic field, for instance provided by one or more magnets. The second metal screening stage may remove magnetic materials from the waste material. Preferably the second metal screening stage is provided after the shredding stage. Preferably the second metal screening stage is provided before a first liquid application stage.

The application of the salt may be provided by a first liquid application stage. The first liquid application stage may apply the salt to the waste material. The first liquid application stage may be provided after the shredding stage and/or second magnetic screening stage. The first liquid application stage may be provided before the dewatering stage.

The salt is preferably absorbed by the SAP. The salt may impede the SAP's ability to bond with water molecules, preferably thereby providing the deactivation. The salt may deactivate the SAP and reduce its capacity and/or prevent it from retaining the liquid it has absorbed. The liquid is therefore able to be substantially and/or fully removed from the SAP when the waste is dewatered, resulting in a dry and biologically sanitised material.

The salt used is preferably an alum, which may be aluminium sulphate. Preferably the aluminium sulphate is applied to the waste material as a solution and preferably a saturated solution. This assists in absorption of the aluminium sulphate by the SAP, with the use of a saturated solution maximising the efficacy in deactivating the SAP.

Preferably no water or other liquid is added to the waste material between the exit from the shredding stage and the waste material entering the first liquid application stage. Preferably no water or other liquid is added to the waste material between the exit from the shredding stage and the waste material exiting the first liquid application stage, other than the salt containing liquid.

Preferably the process provides for no water or liquid being added to the waste material between entry to the process and exit from the process, other than in the first liquid application stage and/or the second liquid application stage.

The biocide may be applied by a liquid application stage, particularly a second liquid application stage. The second liquid application stage may apply the biocide to the waste material. The second liquid application stage may be provided after the dewatering stage. The second liquid application stage may be provided before the waste material wrapping stage.

The liquid biocide may be absorbed by the SAP and renders the SAP biologically safe. The liquid biocide may decontaminate any biological material on the surface of the waste material.

The biocide may be an active germicide such as Envirosan®. The liquid biocide is preferably sprayed onto the waste material. A misting apparatus is used to spray the biocide. Spraying the material in this way optimises coverage of the material and allows the liquid to be applied in an even and equal distribution. Whilst the biocide and aluminium sulphate may be sprayed into the waste material simultaneously, it is preferred that they are applied in separate stages.

The salt is preferably a liquid aluminium sulphate solution. The volume of liquid aluminium sulphate solution applied to the waste material is selected to fully saturate the super absorbent material. This ensures that the SAP is completely deactivated to ensure full dewatering.

The volume of liquid aluminium sulphate solution applied to the waste material may be selected to exceed the absorptive volume of the super absorbent polymer. This ensures full saturation. As the waste material is dewatered after this stage, any excess liquid remaining on the material is removed and does not present a problem. The volume selected to exceed the absorptive volume of the super absorbent polymer may be the combined volume of the liquid aluminium sulphate solution and any other liquid applied, such as a liquid biocide.

A dosing period is preferably allowed between the application of salt to the waste material and the dewatering stage. The term dosing period means a period for which the waste material is allowed to soak up the liquid that has been applied to it. The dosing period is selected to allow the super absorbent polymer to become fully saturated with the salt before dewatering.

The waste material is preferably held on a bulk waste conveyor for the dosing period before being transferred to the dewatering stage.

The dewatering of the waste material may be provided by a dewatering stage. The dewatering stage may comprise compressing the waste material and/or using a screen element and/or filter element to separate the solid waste matter from the liquid removed under compression. Preferably the waste material is compressed by a dewatering screw press, which it has been found provides a highly efficient means of dewatering bulk waste material of this type.

The dewatering screw press may provide a rotating shaft with a helical thread, provided within an enclosure. The volume between the shaft, helical thread and enclosure may be reduced towards the outlet end of the dewatering screw press. The volume reduction preferably causes compression of the waste material. The compression of the waste material causes the liquid to exit through apertures in the enclosure, particularly the lower part thereof, most preferably under the action of gravity. The liquid forms a waste liquid stream.

In an alternative form, the dewatering screw press may include one or more of the following features:
Feeding the waste material via an enclosed conveyor, such as a screw conveyor, to the dewatering screw press;
Feeding the waste material into the dewatering screw press using a chute;
Conveying the waste material away from the entry to the dewatering screw press by means of a rotating shaft and/or helical screw thread;
Conveying the waste material towards a pressure application location within the dewatering screw press, for instance at the end of the dewatering screw press away from the entry;
Applying pressure to the waste material at the pressure application location, for instance by means of one or more elements and/or the rotating shaft and/or helical thread;
The one or more elements being two hydraulically operated pressure applying flaps;
The one or more elements being moveable, for instance closer together or further apart, for instance to increase or decrease the pressure applied to the waste material;
The one or more elements may oppose the spreading of the waste material which is driven between them by the rotating screw;
Applying the pressure causes a plug of waste material to form;
The dewatered waste material being extruded continuously from the dewatering stage or being removed in batches;
The liquid from the dewatering draining out through the bottom of the dewatering stage to a separate location, for instance for further treatment;
The position of the one or more elements and/or the pressure applied to the one or more elements may controlled via a control panel and can be increased or decreased to achieve a given reduction in moisture content and/or given level of dryness required for the waste material.

Preferably the dewatering stage is conducted at the ambient temperature of the process environment +/−10° C., more preferably +/−5° C. Preferably no heating is inputted to the dewatering stage. Preferably no heating is inputted to the waste material between entry to the dewatering stage and exiting the waste material collection and/or waste material wrapping stage.

The waste material exiting the dewatering stage may have a moisture content of less 40% by weight, more preferably less than 38%, for instance between 30 and 35%. The waste material exiting the dewatering stage may be provided to the second liquid application stage. The second liquid application stage preferably applies the biocide to the waste material.

The waste material from the dewatering stage, preferably after the second liquid application stage, is fed to a waste material collection stage. The waste material collection stage may include forming the waste material into bales and/or wrapping the waste material. The waste material, for instance formed into bales, may be used as a solid fuel.

From the dewatering stage the liquid waste stream may be fed to a liquid waste treatment stage. The liquid waste treatment stage may provide clarification of the liquid waste. The liquid waste treatment stage may provide a dissolve air flotation unit. The liquid waste treatment stage may provide a liquid output which can be discharged direct to the environment.

In another embodiment of the invention, two or more different waste material types may be provided to the waste material reception stage and/or shredding stage. One or more of the different waste material types may pass through a pre-processing stage for that waste. The one or more different waste material types may include soft non-infectious clinical waste. The soft non-infectious clinical waste may include one or more of: tissues, swabs, pads, dressings, paper and plastics from dentists, nurses, doctors and hospitals. The pre-processing stage may provide sterilisation of the waste stream type, preferably before reaching the shredder stage. The sterilisation may be achieved through the application of heat, chemicals, pressure and/or irradiation, either singularly or in various combinations. Once sterilised, the waste stream can simply be fed to the waste feed or it may be mixed and blended into the one or more other feed waste types, for instance to create a more homogeneous feed.

In another embodiment of the invention, one or more or all stages of the process may be conducted in a process environment which is isolated from the surrounding environment.

The waste material may be conveyed to the process environment in sealed bags or other containers, ideally which prevent odour release from the untreated waste material.

The process environment may be defined by one or more buildings. The process environment may be defined by the walls, doors and roofs of the structures, for instance to prevent the movement of the air around the waste material in the process environment passing into the surrounding environment, potentially considered as outside.

The process environment may contain one or more or all of: the waste material reception stage; the first metal screening stage; the shredding stage; the second metal screening stage; the first liquid application stage; the dosing stage; the dewatering stage; the second liquid application stage; the waste material collection stage; the liquid waste treatment stage.

Preferably air in or entering the process environment passes to an air treatment stage before exiting the process environment. The air treatment stage may include air treatment units provided at the exit for the air from the process environment. The air treatment units consist of fans to draw the air into the air treatment units from the environment within the facilities. In the air treatment units, sterilisation and/or odour removal steps may be provided, for instance using UV and/or ozone treatment of the air. The sterile and/or de-odourised air then leaves the process environment and enters the surrounding environment after treatment.

### BRIEF DESCRIPTION OF THE DRAWINGS

The present invention will now be described by way of example only with reference to the following illustrative figures in which:

FIG. 1 is a schematic of a method waste material processing according to a first embodiment of the present invention;

FIG. 2 is a schematic of a method of waste material processing according to a second, preferred, embodiment of the present invention.

### DETAILED DESCRIPTION OF THE PREFERRED EMBODIMENTS

### First Embodiment Process

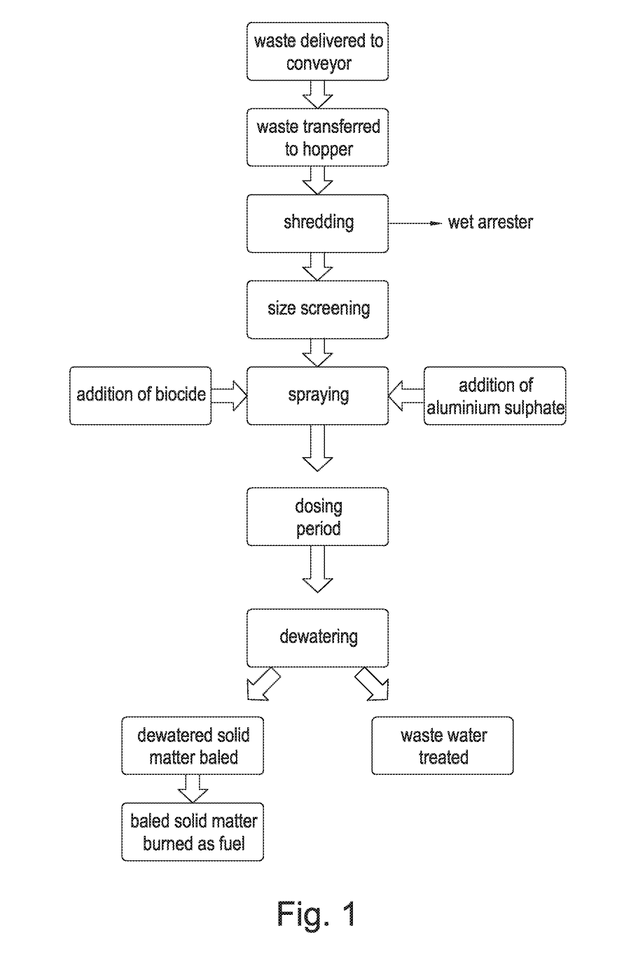

Referring to FIG. 1, a process is described for the processing of absorbent sanitary was material. Such material is classified under European Waste Code 18 01 04 and 20 01 99 and may include incontinence products, nappies and sanitary towels from washroom environments.

The waste material may be collected in bulk by a waste management service provider directly from bulk users such as nursing and residential homes, children's nurseries and washrooms. In addition, or alternatively the waste may be generated from municipal waste collections. The waste material may include any absorbent sanitary products including, but not limited to, disposable nappies, feminine hygiene products, pulp fibre bedpans, slipper pans and urinals, incontinence pads and other products.

At the start of the process waste is initially received and unloaded and visually inspected to identify and remove non-compliant items. The raw waste is then transferred to a bulk hopper which holds and contains the waste in its unrefined condition. Transfer may be by load handling vehicle such as a tele-handler having a loading bucket. At this stage the waste is in the unprocessed form in which it was collected.

The hopper channels the waste to its outlet and on to an infeed conveyor which transfers the waste to a shredder. A hydraulic feed ram forces the material into the shredder. The shredder comprises one or more spinning rotor shafts including cutting blades arranged along its length that rotate with the rotor. The cutting blades are spaced and sized to shred the waste material to a pre-determined particle size. It has been found by the applicant that a preferable particle size for processing is a shredded particle shred size no greater than 60 mm. To ensure uniform particle size the shredded waste material is passed through a size selective screen section. For a selected maximum shredded particle size of 60 mm the screen is a hexagonal screen having a pass size of 80 mm. At this particle size plastic, SAP and fibrous material is passed through the screen. A wet arrester is utilised to draw off any steam generated during the shredding process and to condensate.

The shredded material is then transferred to a treatment or ‘dosing’ conveyor. While on the conveyor the waste material is sprayed directly via direct spray nozzles with both a salt solution and a biocide.

The biocide is sprayed by a nozzle configured to generate a mist. The purpose of spraying the biocide at this stage of the process if to effectively treat and render biologically safe all processed material. The biocide Dilution ratio is preferably 1 to 9 (2lt Biocide to 18lt H₂O). The biocide composition is preferably an active germicide, and is provided at a biocide dilution ratio of 1 to 9 (2lt Biocide to 18lt H₂O).

The salt solution is preferably aluminium sulphate, which is added to the waste material to deactivate the SAP held within the input material. The aluminium sulphate may be added at a dilution level of <10% by weight. The aluminium sulphate is sprayed in a liquid form. The liquid aluminium sulphate is supplied as a saturated solution with the empirical formula:

Al₂(SO₄)₃.51.8 H₂O,

which may be alternatively expressed as:

Al₂O₃.3SO₃.51.8 H₂.

The Al₂O₃ content of the product is 8% w/w and is referred to as ‘8% Aluminium Sulphate’. In Aluminium terms the product contains 4.2% w/w, and as Aluminium Sulphate it contains 26.8% w/w.

The aluminium sulphate and biocide that is sprayed onto the waste along the dosing conveyor by the misting system is absorbed by the SAP within the waste. The volume of biocide and aluminium sulphate sprayed onto the waste material is selected to ensure the SAP material within the waste becomes completely saturated with biocide and aluminium sulphate. Preferably this is achieved by selecting the sprayed volume of liquid to exceed the absorptive capacity of the SAP within the waste material. The sprayed waste material is conveyed and retained on a bulk conveyor for a dosing period that allows the sprayed liquid to be absorbed by the waste material. The waste material may also be agitated while on the conveyor to assists in absorption. The waste material is preferably retained on the slow moving bulk conveyor for a dosing period of approximately 5 minutes. The biocide absorbed by the SAP during this period sterilises the material to make it safe for onward use. At the same time, the aluminium sulphate absorbed by the SAP material deactivates the SAP and prevents it from retaining liquid.

Following the dosing stage, the treated material is fed via an enclosed transfer screw conveyor directly to a dewatering screw press. The dewatering screw press is used to separate the liquid fraction (effluent) from the solid waste material, referred to herein as the dry fraction. The dry fraction includes the shredded plastic material, SAP, pulp fibres and any other solid material used to form the sanitary products being processed.

The dewatering screw press separates the dry fraction from the liquid fraction by compressing the waste matter against a perforated screen to extract the liquid and retain the solid matter. The screw press comprises a rotating shaft around which is provided with an interrupted screw thread. A cylindrical perforated screen surrounds the shaft, having a diameter equal to the diameter of the screw thread. The perforations may have a diameter of approximately 1.5 mm. As the shaft rotates, waste material is held between the screw threads by the screen. The pitch of the thread reduces along the length of the shaft in the direction of travel, which axially compacts the waste material. In addition, the diameter of the shaft increases in the direction of travel, which reduces the spacing between the shaft and the screen, thereby radially compacting the waste material. As that waste material is axially and radially compacted, the liquid fraction is forced out and passes through the perforations in the screen.

As the SAP has been deactivated in the previous dosing stage, the SAP does not retain any liquid, and it is possible to fully dewater all of the waste material as the material is squeezed against the perforated screen system. All of the liquid fraction is forced out through the screen, and the dry fraction, including the SAP, is retained within the screw press.

The compacted waste material exits the dewatering screw press and is transferred from the dewatering screw press to a waste baler for final processing into bales of waste product. The waste baler further compacts and shapes the dewatered waste material into uniform tied off bales that are easily handled, stored and transported. An approximate bale size may be in the region of 1.2 m³, with a weight of up to 1 tonne.

The waste material is loaded onto a conveyor from the dewatering screw press and fed into a feeding chute on the horizontal baler. The press cycle is activated by a light-sensitive barrier integrated into the feeding chute. The speed of the press-ram is controlled by the pressure valves. The overall pressing cycle is controlled and adapted to the pressure, time and distance parameters. The press-ram is retracted automatically to the basic position on completion of the press cycle and the pressing cycle restarts until a complete bale is formed.

The baled material is finally wrapped using a polythene system ready for use as a refuse derived fuel (RDF). Enclosing the bales in polythene makes transportation easier and also allows the fuel bales to be stored for long periods of time without the production of leachate or odours, both of which are contained using the plastic film. The fuel bales in this form may be delivered to energy production facilities where they may be immediately used as fuel, for example for the production of electricity, without the requirement for any additional processing.

The liquid fraction extracted from the waste matter is further treated to remove any residual small particulate suspended solid matter. Finer grade filters and perforated screens are used to remove these remaining small particles of fibre and plastics that may have entered the effluent from the dewatering screw press. The filtered liquid is then diluted in large liquid holding tanks using locally sourced and filtered water to allow for final discharge as trade effluent.

### Second Embodiment Process

The second embodiment process is an improved version of the first embodiment process. As such, many of the features, options and possibilities for the first embodiment are also applicable to the second embodiment. In the description of the second embodiment, emphasis is placed on the differences compared with the first embodiment process.

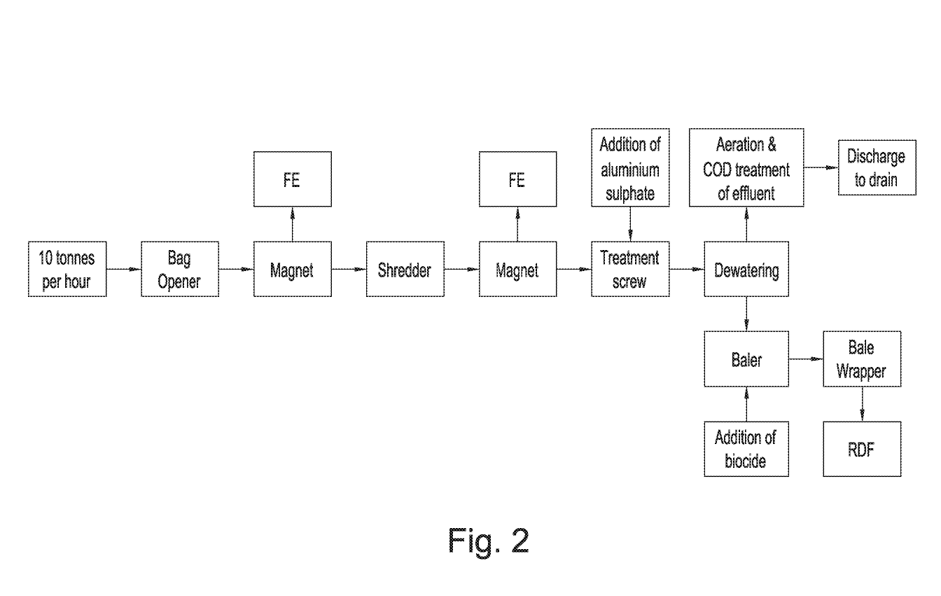

Referring to FIG. 2, the process is concerned with the processing of the same types of absorbent sanitary waste material as the first embodiment.

At the start of the process, the waste is initially received in a bag or other container which is opened to access the waste. The waste is loaded onto an open top conveyor and passes through a first metal screening step where a magnet is used to remove ferrous and other magnetic material in the waste stream. These elements are removed from the waste stream to avoid any deleterious impact on the subsequent processing steps.

The feed conveyor then continues, carrying the waste to a shredder. In the preferred second embodiment, the shredder comprises a dual shaft shredder in which two counter rotating shafts are arranged in proximity with one another with their rotational axis parallel to each other. Each shaft is provided with a series of cutting blades with the cutting blades on one shaft spaced axially so that they are opposite a gap between cutting blades on the other shaft. In this manner, the cutting blades are interdigitated to provide effective shredding the shafts and more particularly the cutting blades are spaced and sized to shred the waste material to a pre-determined particle size. It has been found by the applicant that a preferable particle size for processing is a shredded particle shred size no greater than 120 mm. Typical particles are sections of the waste material with a longest dimension, length, of 120 mm or less, a smallest dimension, thickness, of less than 10 mm and a dimension perpendicular to the smallest dimension and coplanar or perpendicular to the longest dimension, a width, of less than 32 mm. The configuration and operation of the dual shaft shredder is very reliable in providing a given maximum size and so no screening or other size verification step is necessary. The avoidance of the need for a screen reduces the chances of production disrupting blockages on the screen.

The shredded material is then transferred to second conveyor. Again, the second conveyor is open topped and passes through a second metal screening step where a magnet is used to remove ferrous and other magnetic material in the waste stream. These elements are removed from the waste stream to avoid any deleterious impact on the subsequent processing steps. The second metal screening step is particularly provided to remove any magnetic material released by the shredding step and/or rendered more exposed in the finer waste material after shredding.

The second conveyor continues and provides the waste material to a treatment or ‘dosing’ step. While on the conveyor the waste material is sprayed directly via direct spray nozzles with a salt solution. No other liquid or solid materials are applied to the waste in this step. The biocide mentioned in this step in the first embodiment is now provided at a later step in the process.

The salt solution is preferably aluminium sulphate as with the first embodiment.

Following the dosing stage, the treated material is fed via an enclosed transfer screw conveyor directly to a dewatering stage. The stage includes a dewatering screw press, but differs from the first embodiment in which the diameter of the shaft increasing in the direction of travel to reduce the space and so compress the waste material further. In the second embodiment, a chute feeds the waste material into the dewatering screw and its chamber, from above. The waste material is conveyed by the rotating screw of the dewatering screw press away from the entry and towards a counter pressure unit at the other end. The counter pressure unit is formed of two hydraulically operated pressure applying flaps. These can be moved within an enclosing chamber, closer together or further apart. The flaps oppose the spreading of the waste material which is driven between them by the rotating screw. This causes a plug of waste material to form. The flaps can be moved closer together and/or be inclined closer together as the waste material progresses further away from the entry so as to increasingly dewater the waste material. The dewatered plug can be extruded continuously from the dewatering stage, with the liquid draining out through the bottom of the stage to a separate location, or in batches.

The position of the flaps and/or the pressure applied to the flaps is controlled via a control panel and can be increased or decreased to achieve a given reduction in moisture content and/or given level of dryness required for the waste material. Differences in dewatering characteristics for different batches of waste material can also be accommodated in this manner and yet achieve a consistent waste material after dewatering.

The compacted waste material which exits the dewatering screw press is again transferred a waste baler step for final processing into bales of waste product.

Prior to, or within the waste baler step, a biocide is sprayed by a nozzle configured to generate a mist. The purpose of spraying the biocide at this stage of the process is to effectively treat and render biologically safe all processed solid material. Adding the biocide at this step is preferable as it prevents biocide being present in the liquid effluent generated in the dewatering step. This is an issue with the first embodiment. The properties of the biocide are as per the first embodiment.

The waste baler further compacts and shapes the dewatered waste material into uniform tied off bales that are easily handled, stored and transported, as with the first embodiment. The water content of the waste in the bales is around 30-35% moisture by weight and as a consequence the waste is light and fluffy in consistency.

The baled material is finally wrapped using a polythene system ready for use as a refuse derived fuel (RDF), as with the first embodiment.

The liquid fraction extracted from the waste matter is further treated to remove any residual small particulate suspended solid matter using a dissolved air flotation unit. Air is dissolved in the liquid under pressure and on entering a flotation tank forms air bubbles as the pressure is released. The air bubbles adhere to the particulate material and rise to the surface of the tank where they are skimmed off. The result is a clarified liquid fraction which is clean enough to be discharged as waste water. In extreme cases, some chemical treatment, for instance flocculation, may be required to remove difficult to extract solids.

### Third Process Embodiment

In the case of either of the first or second process embodiments described above, it is possible to accommodate other forms of waste feed material through the provision of a pre-processing stage for that waste. The forms of waste described in the first and second embodiments do not need to pass through this additional pre-processing stage.

The pre-processing stage is used to accommodate more problematic waste streams such as soft non-infectious clinical waste. This waste is exemplified by tissues, swabs, pads, dressings, paper and plastics from dentists, nurses, doctors and hospitals which have been used, but which are free of infection risk. Due to the potential risks from such wastes, however, the third embodiment provides for the sterilisation of this waste stream before entering the process described above.

The sterilisation may be achieved through the application of heat, chemicals, pressure and/or irradiation, either singularly or in various combinations. For instance, heat based sterilisation may employ autoclaves, ovens or the like. For instance, chemical based sterilisation may employ ethylene oxide, nitrogen dioxide, ozone or other agents. For instance, irradiation based sterilisation may be provided by non-irradiating sterilisation (such as UV application) in preference to ionising irradiation.

Once sterilised, the waste stream can simply be fed to the waste feed to the first and/or second embodiment process or it may be mixed and blended into the normal feed waste for that process so as to create a more homogeneous feed.

### Environment Control for the Process Embodiments

Due to the nature and the volume of the waste being processed, steps are taken to prevent odours leaking from the process environment into the surrounding environment. This involves the waste being conveyed to the processing site in sealed bags or other containers which prevent odour release from the untreated waste. Once on site, the sealed bags continue to prevent odour release, but in a preferred format the waste is stored in enclosed storage facilities before moving to the process plant. The process plant too is provided within an enclosed process facility. The enclosed storage facility and/or process facility are formed by walled and roofed structures which prevent the movement of the air around the waste into the general environment. Whilst access doors are provided to allow access to the facilities, these are only open when necessary. Furthermore, even when open air is drawn in through these openings and then passes to air treatment units provided at the exit for the air from the facilities.

The air treatment units consist of fans to draw the air into the air treatment units from the environment within the facilities. In the air treatment units, sterilisation and/or odour removal steps are provided, for instance using UV and/or ozone treatment of the air. The sterile and/or de-odourised air then leaves the facility and enters the surrounding environment.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A method of processing waste material including a super absorbent polymer, the method comprising:
shredding a waste material comprising a super absorbent polymer;
applying a liquid biocide to the shredded waste material;
applying a salt to the shredded waste material to deactivate the super absorbent polymer; and
dewatering the waste material following deactivation of the super absorbent polymer.

2. The method according to claim 1, wherein the salt is aluminium sulphate.

3. The method according to claim 1, wherein the step of applying a liquid biocide to the shredded waste material is provided after the step of dewatering the waste material.

4. The method according to claim 1, wherein no water or other liquid is added to the waste material between the start of the processing and the waste material exiting a shredding stage which provides the shredding of the waste material.

5. The method according to claim 1, in which the moisture content of the waste material, excluding the moisture absorb by the super absorbent polymer in the waste material, is less than 25% by weight on entering the shredding stage and/or on exiting the shredding stage.

6. The method according to claim 1, wherein the method provides, in sequence: a shredding stage; a first liquid application stage; a dewatering stage; a second liquid application stage.

7. The method according to claim 1, wherein the method provides, in sequence: a waste material reception stage; a first metal screening stage; a shredding stage; a second metal screening stage; a first liquid application stage; a dosing stage; a dewatering stage; a second liquid application stage; a waste material collection stage.

8. The method according to claim 2, wherein the aluminium sulphate is applied to the waste material as a solution.

9. The method according to claim 2, wherein the aluminium sulphate solution is a saturated solution.

10. The method according to claim 1, wherein the biocide is an active germicide.

11. The method according to claim 1, wherein the liquid biocide is sprayed onto the waste material.

12. The method according to claim 1, wherein the salt is a liquid aluminium sulphate solution and is sprayed onto the waste material.

13. The method according to claim 1, wherein the salt is a liquid aluminium sulphate solution and the volume of liquid aluminium sulphate solution applied to the waste material is selected to fully saturate the super absorbent material.

14. The method according to claim 13, wherein the volume of liquid aluminium sulphate solution applied to the waste material is selected to exceed the absorptive volume of the super absorbent polymer.

15. The method according to claim 1, wherein the dewatering stage comprises compressing the waste material and using a screen element and/or filter element to separate the solid waste matter from the liquid removed under compression.

16. The method according to claim 15, wherein the waste material is compressed by a dewatering screw press.

17. The method according to claim 1, wherein the dewatering of the waste material is provided by a dewatering screw press which include one or more of the following features or steps:
conveying the waste material away from the entry to the dewatering screw press by means of a rotating shaft and/or helical screw thread;
conveying the waste material towards a pressure application location within the dewatering screw press, for instance at the end of the dewatering screw press away from the entry;
applying pressure to the waste material at the pressure application location, for instance by means of one or more elements and/or the rotating shaft and/or helical thread;

18. The method according to claim 1, wherein the dewatering of the waste material in a dewatering stage is conducted at the ambient temperature of the process environment +/−10° C.

19. The method according to claim 1, wherein no heating is inputted to the dewatering stage provided to dewatering of the waste material.

20. The method according to claim 1, wherein no heating is inputted to the waste material between entry to the dewatering stage and exiting the waste material collection and/or waste material wrapping stage.

21. The method according to claim 1, further including compacting the waste material into bales following the dewatering stage.

22. The method according to claim 1, wherein the dewatered waste material is used as a solid fuel.

23. The method according to claim 1, wherein the shredding stage is conducted at the ambient temperature of the process environment +/−10° C.

24. The method according to claim 1, wherein the process and its stages are all conducted at the ambient temperature of the process environment +/−10° C.

25. The method according to claim 1, wherein no heating is inputted to the waste material between entry to the process and exiting the process.

26. The method according to claim 1, wherein no separation of a plastics content of the waste material from the super absorbent polymer content of the waste material is provided or sought in the process.

27. The method according to claim 1, wherein a dosing period is allowed between the application of the salt to the waste material and the dewatering stage that is selected to allow the super absorbent polymer to become fully saturated with the salt before dewatering.

28. The method according to claim 27, wherein the waste material is held on a bulk waste conveyor for the dosing period before being transferred to the dewatering stage.
